(19) United States
(12) Patent Application Publication
(10) Pub. No.: US 2026/0346023 A1
(43) Pub. Date: Nov. 5, 2026

(54) IMPLANTABLE MEDICAL STIMULATION DEVICE AND METHOD FOR PROVIDING AN ATRIAL ANTITACHYCARDIA PACING THERAPY

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 19/116,262

(22) PCT Filed: Jul. 21, 2023
(86) PCT No.: PCT/EP2023/070339

§ 371 Date: Mar. 27, 2025

(30) Foreign Application Priority Data
Oct. 7, 2022 (EP) 22200214.9

(51) Int. Cl. A61N 1/362; A61B 5/00; A61B 5/363; A61N 1/365

(52) U.S. Cl. CPC A61N 1/3624; A61B 5/363; A61B 5/4836; A61B 5/686; A61N 1/36514

(57) ABSTRACT

An implantable medical stimulation device for providing an atrial antitachycardia pacing therapy includes a generator device comprising processing circuitry for processing cardiac sense signals to detect an atrial tachycardia and to generate stimulation signals for providing an atrial antitachycardia pacing therapy. The processing circuitry is configured to store a reference value based on a measure indicative of the atrial interval following a detection of an atrial antitachycardia and prior to triggering a first atrial antitachycardia therapy attempt, to compare a value of said measure indicative of the atrial interval subsequent to the first atrial antitachycardia therapy attempt to said reference value, and to trigger a second atrial antitachycardia therapy attempt if a difference between said value and said reference value is equal to or larger than a predefined margin. The predefined margin is equal to a predefined percentage of said reference value.

### CROSS-REFERENCE TO RELATED APPLICATIONS

This application is the United States National Phase under 35 U.S.C. § 371 of PCT International Patent Application No. PCT/EP 2023/070339, filed on Jul. 21, 2023, which claims the benefit of European Patent Application No. 22200214.9, filed on Oct. 7, 2022, the disclosures of which are hereby incorporated by reference herein in their entireties.

### TECHNICAL FIELD

The instant invention relates to an implantable medical stimulation device for providing an atrial antitachycardia pacing therapy according to the preamble of claim 1 and to a method for operating an implantable medical stimulation device.

### BACKGROUND

An implantable medical stimulation device of this kind comprises a generator device having processing circuitry for processing cardiac sense signals to detect an atrial tachycardia and to generate stimulation signals for providing an atrial antitachycardia pacing therapy.

Atrial antitachycardia pacing (aATP) serves to overcome an atrial tachycardia (AT), such as an atrial fibrillation (AF). Implantable medical stimulation devices, such as implantable cardioverter defibrillator (ICD) devices, implement protocols for antitachycardia pacing therapies that are automatically applied upon detection of atrial tachyarrhythmias.

For providing an atrial antitachycardia pacing, generally an atrial tachycardia is detected by monitoring an atrial rhythm. If it for example is found that an atrial interval becomes short and falls into a tachycardie zone (e.g. below a predefined tachycardia threshold), it is assumed that an atrial tachycardia is present, and accordingly an atrial antitachycardia pacing therapy is initiated.

In the context of an atrial antitachycardia pacing therapy one or multiple bursts or sequences of bursts are generated at a rate larger than the tachycardie rhythm in order to reset the atrial rhythm to a normal, non-tachycardie rhythm. Antitachycardia pacing (ATP) has been recognized as a way to terminate arrhythmias by delivering stimulation signals to the heart at a rate faster than the tachycardia in order to pace into an excitable gap between successive activations when the myocardium is susceptible to respond to excitations and in this way terminate an existing tachycardia.

In the presence of an atrial tachycardia, an atrial antitachycardia pacing therapy may be preferable over delivery of a defibrillation shock therapy, in that antitachycardia pacing generally can be assumed to be not painful to the patient and may reduce battery drainage. However, although an atrial antitachycardia therapy allows for a termination of an atrial tachycardia, in certain scenarios an antitachycardia pacing therapy may have a limited chance of success. It hence is advisable to program a stimulation device such that an atrial antitachycardia pacing therapy is repeated only if a chance of success has changed, and to refrain from a repetition if the chance of success has not changed.

For example, in an approach described in U.S. Pat. No. 6,876,880 an atrial interval is monitored, and according to the atrial interval an atrial tachycardia is classified to fall into a so-called bin out of a multiplicity of predefined bins. Following an atrial antitachycardia pacing therapy attempt another antitachycardia pacing therapy attempt is triggered once the atrial interval is classified to fall into another bin, i.e., when the atrial interval has changed such that it now falls into another bin in comparison to the previous bin. This is based on the assumption that a particular antitachycardia pacing therapy may initially be unsuccessful in terminating the atrial tachycardia, but may prove successful when the atrial interval, also denoted as cycle length, or regularity of the atrial rhythm changes.

The present disclosure is directed toward overcoming one or more of the above-mentioned problems, though not necessarily limited to embodiments that do.

### SUMMARY

It is an object of the instant invention to provide an implantable medical stimulation device and a method for operating an implantable medical stimulation device for providing for an antitachycardia pacing which allow, in an easy and efficient way, to reapply an antitachycardia pacing therapy with improved chance of success while avoiding unnecessary reapplications of therapy attempts.

This object is achieved by means of an implantable medical stimulation device comprising the features of claim 1.

In one aspect, an implantable medical stimulation device for providing an atrial antitachycardia pacing therapy comprises: a generator device comprising processing circuitry for processing cardiac sense signals to detect an atrial tachycardia and to generate stimulation signals for providing an atrial antitachycardia pacing therapy. The processing circuitry is configured to monitor a measure indicative of an atrial interval, to detect an atrial tachycardia based on said measure indicative of the atrial interval, and to trigger a first atrial antitachycardia therapy attempt if an atrial antitachycardia is detected. The processing circuitry is configured, subsequent to said first antitachycardia therapy attempt, to further monitor said measure indicative of the atrial interval and to evaluate whether said atrial antitachycardia persists. The processing circuitry is configured to store a reference value based on said measure indicative of the atrial interval following said detection of the atrial antitachycardia and prior to triggering said first atrial antitachycardia therapy attempt, to compare a value of said measure indicative of the atrial interval subsequent to said first atrial antitachycardia therapy attempt to said reference value, and to trigger a second atrial antitachycardia therapy attempt if a difference between said value and said reference value is equal to or larger than a predefined margin. The predefined margin is equal to a predefined percentage of said reference value.

The implantable medical stimulation device comprises a generator device comprising processing circuitry which serves to process cardiac sense signal to detect an atrial tachycardia and to generate stimulation signals for providing an atrial antitachycardia pacing therapy. The implantable medical stimulation device may for example be an implantable pulse generator (IPG), an implantable cardioverter defibrillator (ICD) or a cardiac resynchronization therapy (CRT) device, such as a CRT-D or CRT-P device.

The implantable medical stimulation device may for example be configured for a subcutaneous implantation, in which the generator device is implanted subcutaneously in a patient and one or multiple leads carrying electrode pole arrangements extend from the generator device into the patient's heart in order to sense cardiac signals from the patient's heart and to generate and inject stimulation signals into the patient's heart.

The processing circuitry is configured to monitor a measure indicative of an atrial interval in order to detect an atrial tachycardia based on the measure indicative of the atrial interval. The measure indicative of the atrial interval may correspond to a time span in between successive atrial peaks, wherein the measure may be determined for example in a single cycle or by averaging over atrial intervals in multiple cycles.

If an atrial tachycardia is detected, for example by comparing the measure indicative of the atrial interval to a detection threshold in order to determine whether the atrial interval lies within a tachycardie zone, a (first) atrial antitachycardia therapy attempt is triggered. Generally, within an atrial antitachycardia therapy attempt one or multiple bursts or sequences of bursts may be generated by the generator device and output by the implantable medical stimulation device at a rate higher than the tachycardie rate in order to attempt to terminate the atrial tachycardia.

Subsequent to the (first) atrial antitachycardia therapy attempt it is monitored whether the atrial antitachycardia therapy attempt has been successful. For this, it is evaluated whether the atrial antitachycardia persists, i.e., whether the atrial interval still lies within the tachycardie zone, for example below an atrial tachycardie threshold, or not. If the atrial interval is no longer tachycardie, it is assumed that the atrial antitachycardia therapy attempt has been successful. If instead the atrial interval still is tachycardie, it is assumed that the atrial tachycardia persists.

If the first atrial antitachycardia therapy attempt has not been successful, it is evaluated whether a different tachycardie situation arises and a following, second atrial antitachycardia therapy attempt hence may have a different chance of success. This is based on the assumption that, without a significant change in the tachycardie situation, a mere repetition of atrial antitachycardia therapy attempts poses a burden on the patient and the system without however having a substantially increased chance of success in terminating the atrial tachycardia over the first, prior atrial antitachycardia therapy attempt, which has not proven successful. It hence is checked whether the atrial interval has changed by a predefined margin or more than the predefined margin, and only if this is the case another atrial antitachycardia therapy attempt is triggered.

Specifically, the processing circuitry is configured to store a reference value based on the measure indicative of the atrial interval following the detection of the atrial antitachycardia and prior to triggering the first atrial antitachycardia therapy attempt. The reference value shall serve as a reference for the atrial interval at the time of the first atrial antitachycardia therapy attempt. Subsequent to the first atrial antitachycardia therapy attempt, then, the measure indicative of the atrial interval is monitored and compared to the reference value, and another, second atrial antitachycardia therapy attempt is triggered if a difference between a value of the measure indicative of the atrial interval subsequent to the first atrial antitachycardia therapy attempt differs from the reference value by the predefined margin or by more than the predefined margin.

For triggering another atrial antitachycardia therapy attempt it hence is evaluated whether a substantial change in the atrial interval has occurred. Only if the change is larger than the predefined margin, another atrial antitachycardia therapy attempt is triggered. If not, it is assumed that the tachycardie state in the atrium has not substantially changed, at least not in a way such that chances of success for another atrial antitachycardia therapy attempt have substantially improved.

By comparing a current value of the atrial interval to the reference value, which indicates the atrial interval at the time of the first, prior atrial antitachycardia therapy attempt, and by evaluating whether the difference between the current value and the reference value is equal to or larger than the predefined margin, it is determined whether a sufficient distance between the current atrial interval and the prior atrial interval at the time of the first atrial antitachycardia therapy attempt exists. Only if this is the case, another, second atrial antitachycardia therapy attempt is triggered.

In one embodiment, The predefined margin may for example be equal to a time value in between 25 ms to 100 ms.

In another embodiment, the predefined margin is for example a value in between 10% to 50% of the reference value.

In one embodiment, the processing circuitry is configured to derive the measure indicative of the atrial interval by averaging values of a predefined number of atrial intervals in different atrial cycles, for example a number larger than 3, preferably larger than 5, even more preferably larger than 10 atrial intervals. For example, an average over a number in between 6 to 10 successive atrial intervals, i.e., atrial interval values of 6 to 10 successive atrial cycles, may be taken to derive the measure indicative of the atrial interval, such that a running average is computed to derive a current value for the measure.

It is to be noted that the term “first atrial antitachycardia therapy attempt” not necessarily corresponds to an (absolutely) first atrial antitachycardia therapy attempt in the course of providing an atrial antitachycardia therapy. The term “first atrial antitachycardia therapy attempt” shall generally refer to a prior atrial antitachycardia therapy attempt, and the term “second atrial antitachycardia therapy attempt” shall refer to another, subsequent atrial antitachycardia therapy attempt, wherein one or multiple atrial antitachycardia therapy attempts may have taken place prior to the first atrial antitachycardia therapy attempt and one or multiple atrial antitachycardia therapy attempts may take place subsequent to the second atrial antitachycardia therapy attempt.

In one embodiment, the processing circuitry is configured to trigger the second atrial antitachycardia therapy attempt only if an elapsed time subsequent to the first atrial antitachycardia therapy attempt exceeds a predefined waiting period. The waiting period may lie in a range between 1 minute to 20 minutes and serves to provide for a delay in between the prior, first atrial antitachycardia therapy attempt and another, subsequent, second atrial antitachycardia therapy attempt. If the atrial interval changes immediately after or in close temporal proximity subsequent to the first atrial antitachycardia therapy attempt, another atrial antitachycardia therapy attempt is not triggered immediately, but it is waited until the waiting period has elapsed, upon which it is examined if the now current measure for the atrial interval differs from the reference value as obtained prior to the first atrial antitachycardia therapy attempt by an amount equal to or larger than the predefined margin. If this is the case, a second atrial antitachycardia therapy attempt is triggered. Another, second atrial antitachycardia therapy attempt hence is output only if a sufficient delay after the first atrial antitachycardia therapy attempt has elapsed.

In one embodiment, the processing circuitry is configured to trigger the second atrial antitachycardia therapy attempt only if an elapsed time subsequent to the first atrial antitachycardia therapy attempt does not exceed a predefined coagulation time window. This is based on the experience that blood may coagulate during a persistent atrial tachycardia, such that no further therapy should be attempted if the atrial tachycardia lasts over a prolonged period of time larger than the coagulation time window. The coagulation time window may for example lie in a range between 20 hours to 60 hours, preferably 40 to 50 hours, for example at 48 hours. If the coagulation time window has elapsed, no further atrial antitachycardia therapy attempt is triggered, but rather it is assumed that the patient should attend to a physician.

In one embodiment, the processing circuitry is configured to trigger the second atrial antitachycardia therapy attempt only if an elapsed time subsequent to the first atrial antitachycardia therapy attempt exceeds a predefined waiting period and if, at the same time, the predefined coagulation time window has not yet elapsed.

In one embodiment, the processing circuitry is configured to store the value of the measure indicative of the atrial interval at the time of triggering the second atrial antitachycardia therapy attempt as a new reference value, to compare a value of the measure indicative of the atrial interval subsequent to the second atrial antitachycardia therapy attempt to the new reference value, and to trigger another atrial antitachycardia therapy attempt if a difference between the value and the new reference value is equal to or larger than the predefined margin. Subsequent to the second atrial antitachycardia therapy attempt the process, hence, may be continued in that prior to the second atrial therapy attempt a new reference value is obtained, and subsequent to the second atrial antitachycardia therapy attempt a then current value of the measure indicative of the atrial interval is compared to the new reference value. If the difference between the value and the new reference value is larger than the predefined margin, another atrial antitachycardia therapy attempt may be triggered.

The process may be continued over a multiplicity of atrial antitachycardia therapy attempts, wherein another, subsequent atrial antitachycardia therapy attempt is triggered only if the atrial interval has changed, in comparison to the atrial interval at the prior atrial antitachycardia therapy attempt, by an amount equal to or larger than the predefined margin.

It herein may in addition be taken into account whether the current value differs from all previous reference values by a substantial amount, for example by an amount equal to or larger than the predefined margin, such that it is avoided that an atrial antitachycardia therapy attempt is triggered anew at a value for the atrial interval for which already prior an atrial antitachycardia therapy attempt has been triggered unsuccessfully.

In one embodiment, the processing circuitry is configured to monitor a stability measure indicative of a stability of the atrial interval over time and to trigger an atrial antitachycardia therapy attempt based on a change in the stability matter. For example, the processing circuitry may be configured to classify the atrial tachycardia, based on the stability measure, as stable or unstable and to trigger an atrial antitachycardia therapy attempt if the atrial tachycardia changes from stable to unstable or vice versa. For example, for deriving the stability measure the standard deviation of the atrial interval, i.e., the standard deviation of atrial interval values over a multiplicity of atrial cycles, may be determined. By comparing the standard deviation to a stability threshold, the atrial tachycardia may be classified as stable or unstable. For example, when the stability measure is lower than the stability threshold, it may be classified as stable, and if the stability measure is larger than the stability threshold, it may be classified as unstable. If a change from one regime to the other occurs, i.e., from a stable state to an unstable state or from an unstable state to a stable state, another atrial antitachycardia therapy attempt may be triggered, following for example an additional delay period subsequent to a prior therapy attempt.

In another aspect, a method for operating an implantable medical stimulation device for providing an atrial antitachycardia pacing therapy comprises: processing, using a generator device comprising processing circuitry, cardiac sense signals to detect an atrial tachycardia and generating, using the generator device, stimulation signals for providing an atrial antitachycardia pacing therapy; monitoring, using the processing circuitry, a measure indicative of an atrial interval, detecting an atrial tachycardia based on said measure indicative of the atrial interval, and triggering a first atrial antitachycardia therapy attempt if an atrial antitachycardia is detected; further monitoring, using the processing circuitry, said measure indicative of the atrial interval subsequent to said first antitachycardia therapy attempt, and evaluating whether said atrial antitachycardia persists; storing, using the processing circuitry, reference value based on said measure indicative of the atrial interval following said detection of the atrial antitachycardia and prior to triggering said first atrial antitachycardia therapy attempt; comparing, using the processing circuitry, a value of said measure indicative of the atrial interval subsequent to said first atrial antitachycardia therapy attempt to said reference value; and triggering, using the processing circuitry, a second atrial antitachycardia therapy attempt if a difference between said value and said reference value is equal to or larger than a predefined margin. The predefined margin is equal to a predefined percentage of said reference value.

The advantages and advantageous embodiments described above for the implantable medical stimulation device equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea underlying the invention shall subsequently be described in more detail with reference to the embodiments shown in the drawings. Herein:

FIG. 1 shows a schematic drawing of an implantable medical stimulation device for providing an antitachycardia therapy;

FIG. 2 shows a schematic flow diagram of an atrial antitachycardia therapy method;

FIG. 3 shows a schematic diagram of an atrial interval over time; and

FIG. 4 shows an enlarged subsection of the diagram of FIG. 3.

### DETAILED DESCRIPTION

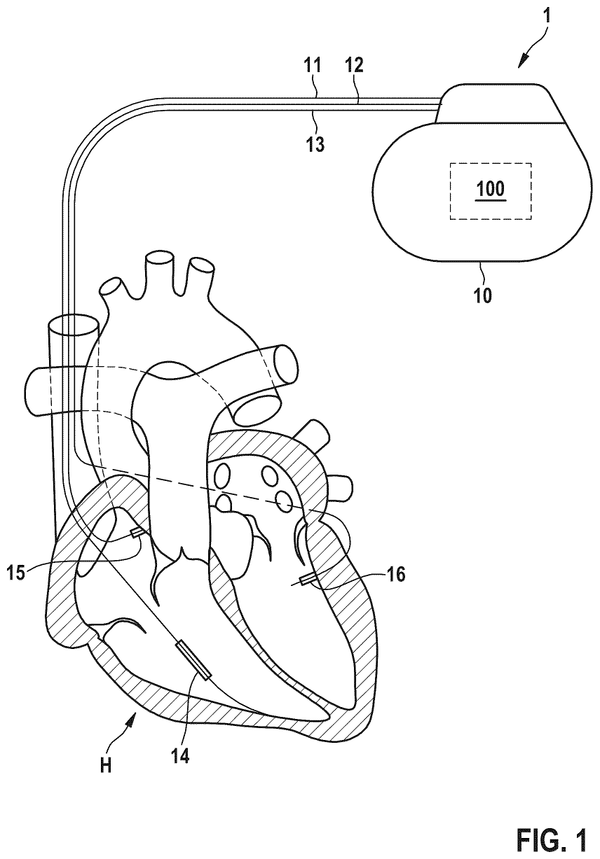

FIG. 1, in a schematic drawing, shows the implantable medical stimulation device, such as a cardiac resynchronization therapy (CRT) device which in addition may comprise a defibrillation function (CRT-D).

It is to be noted that the instant text relates not only to CRT-D devices, but likewise for example to IPG, ICD or CRT-P devices.

The implantable medical stimulation device 1 comprises a generator device 10 which may be for example configured for subcutaneous implantation in a patient. The generator device 10 herein is implanted together with a number of electrode leads 11, 12, 13, which extend from the generator device 10 in order to reach into the patient's heart H. A first lead 11 for example extends into the right ventricle, a second lead 12 for example extends into the right atrium and a third lead 13 for example is placed on the left ventricle. Each lead 11, 12, 13 comprises, in the vicinity of its distal end, an electrode pole arrangement 14, 15, 16, which is configured to sense cardiac sense signals and/or emit stimulation signals in order to provide for a therapy within the patient's heart H.

The electrode pole arrangement 14 of the lead 11 may in addition comprises a shock coil in order to output a shock pulse for achieving a defibrillation action in the right ventricle of the patient's heart H.

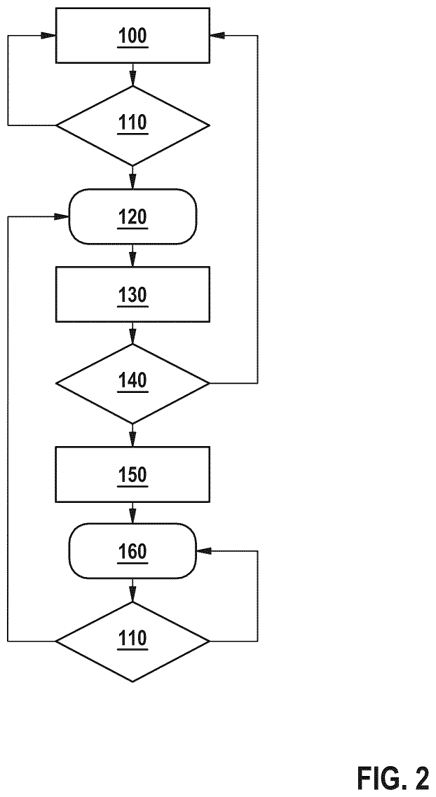

### FIG. 2 shows a flow diagram of an antitachycardia pacing method.

Within the pacing method, in a step 100 an atrial rhythm is monitored, for example by continuously evaluating a measure indicative of an atrial interval in successive atrial cycles. For example, the atrial interval in a current atrial cycle may be determined as the distance between successive peaks relating to atrial activity in a cardiac cycle (so-called p waves in an electrocardiogram signal). To come up with the measure for example an average over multiple atrial cycles may be obtained.

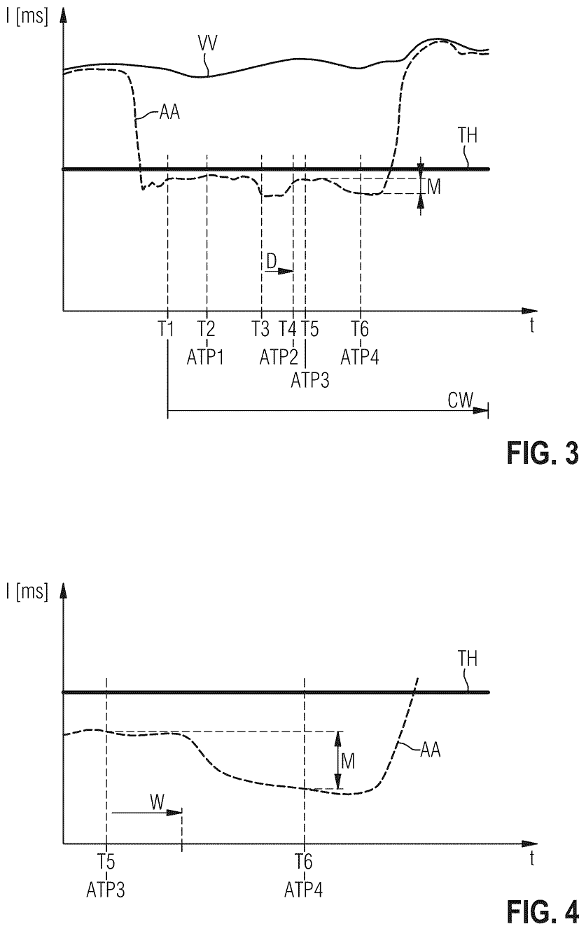

The measure indicative of the atrial interval continuously is compared for example to an atrial tachycardia detection threshold TH (see FIG. 3) in order to evaluate whether be atrial interval falls within a tachycardie zone. Accordingly, in step 110 the atrial rhythm is classified as tachycardic or not.

If, in step 110, it is detected that the measure indicative of the atrial interval indicates an atrial tachycardia, in step 120 a value of the atrial interval at the time of detection of the atrial tachycardia is stored as a reference value. In addition, a stability reference may be stored classifying the atrial tachycardia as stable or unstable for example based on a standard deviation of the atrial interval in a number of successive atrial cycles.

In step 130, subsequent to storing the reference value for the atrial interval and (potentially) in addition the stability reference, a first atrial antitachycardia therapy attempt is triggered, in the course of which one or multiple bursts or sequences of bursts at a rate higher than the tachycardie atrial rate are output by the generator device 10 in order to attempt to terminate the atrial tachycardia.

In step 140 it is evaluated whether the atrial antitachycardia therapy attempt has been successful. If this is the case, the method reverts back to step 100, such that again the atrial rhythm is monitored in order to potentially detect an atrial tachycardia.

If, in step 140, it is found that the first atrial antitachycardia therapy attempt has not been successful, in step 150 a further atrial antitachycardia therapy attempt is blocked for a predefined waiting period. After lapse of the waiting period, in step 160 the atrial rhythm is further monitored. If it is found in step 170 that the atrial rhythm is still tachycardie and the measure indicative of the atrial interval has changed with respect to the reference value as recorded prior to the first atrial antitachycardia therapy attempt by an amount equal to or larger than a predefined margin, another, second atrial antitachycardia therapy attempt is triggered by reverting back to step 120, upon which the cycle of steps 120 to 170 starts anew.

Referring now to FIG. 3, within the implantable medical stimulation device 1 and within a method for operating the implantable medical stimulation device 1 cardiac sense signals are evaluated in order to determine a ventricular interval VV and an atrial interval AA. In a normal state of the patient's heart H, herein, the ventricular interval VV should generally match at least approximately the atrial interval AA.

If, however, an atrial tachycardia is present, the atrial interval AA may drop while the ventricular interval VV remains in a non-tachycardie range. If it is found that the atrial interval AA falls into a tachycardie zone below an atrial tachycardia threshold TH, the atrial rate is classified as an atrial tachycardia, which accordingly may be detected and identified within the processing circuitry 100 of the generator device 10.

For example, at time T1 an atrial tachycardia may be detected. Subsequently, at time T2, an atrial antitachycardia therapy attempt ATP1 is triggered, in the course of which one or multiple bursts or sequences of bursts are output by the generator device 10 in order to attempt to terminate the atrial tachycardia.

Prior to triggering the atrial antitachycardia therapy attempt ATP1 at time T2, a reference value of the atrial interval is stored, and in addition a stability reference may be recorded classifying the atrial tachycardia prior to the atrial antitachycardia therapy attempt ATP1 at time T2 as stable or unstable (in the shown example it is assumed to be stable).

Following the atrial antitachycardia therapy attempt ATP1 at time T2, the atrial rate is continuously monitored in order to evaluate whether the atrial antitachycardia therapy attempt ATP1 has been successful. If it is found that the atrial antitachycardia therapy attempt ATP1 has not been since successful (as in the shown diagram), it is monitored whether the atrial state changes such that a further atrial antitachycardia therapy attempt should be triggered. For this, it is monitored whether the atrial interval changes and/or the atrial tachycardia changes from a stable to an unstable state or vice versa.

In the example of FIG. 3, at time T3 it is found that the atrial tachycardia, which previously has been stable, changes from a stable state to an unstable state. For example, it is found that a stability measure, computed continuously according to for example the standard deviation of the atrial interval over a predefined number of atrial cycles, now is above a stability threshold and hence is indicative of a large variation of atrial interval values in a range of atrial cycles. As at time T3 the stability changes from stable to unstable, at time T4 another atrial antitachycardia therapy attempt ATP2 is triggered, potentially after a delay D in order to not immediately trigger a therapy attempt upon a state change but to allow the atrial rhythm to reset back to its prior, e.g. stable state.

At time T5 it may be identified that the atrial rhythm has reset back to a stable state, but is still tachycardie. At this time, another atrial antitachycardia therapy attempt ATP3 may be triggered.

Subsequent to the atrial antitachycardia therapy attempt ATP3 at time T5 it may again be monitored whether the atrial antitachycardia therapy attempt ATP3 has been successful, and further it may be monitored whether the atrial interval changes by an amount equal to or larger than a predefined margin M with respect to the atrial interval at time T5 of the atrial antitachycardia therapy attempt ATP3.

Referring now to FIG. 4, prior to the atrial antitachycardia therapy attempt ATP3 at time T5 a reference value for the atrial interval and information about the stability state is stored. Specifically, a reference value for the atrial interval and information about the stability state may be stored prior to each atrial antitachycardia therapy attempt ATP1 . . . ATP4.

Following the atrial antitachycardia therapy attempt ATP3, it is evaluated whether the therapy attempt has been successful, and—if it has not been successful—a waiting period W is allowed to lapse.

Upon lapse of the waiting period W the atrial interval is further monitored. If at time T6 it is found that the atrial interval has changed with respect to the atrial interval at the time of the prior atrial antitachycardia therapy attempt ATP3 by an amount equal to or larger than the predefined margin M, another atrial antitachycardia therapy attempt ATP4 is triggered.

Referring now again to FIG. 3, in the shown example subsequent to the atrial antitachycardia therapy attempt ATP4 the atrial tachycardia terminates, such that the atrial antitachycardia therapy attempt ATP4 can be assumed to have been successful in that the atrial interval AA reverts back to a normal, non-tachycardie state.

As illustrated in FIG. 3, as a further condition it may be assessed whether a coagulation window CW has elapsed. An atrial antitachycardia therapy attempt may accordingly be triggered if and only if the coagulation window CW has not elapsed, such that all atrial antitachycardia therapy attempts are terminated upon lapse of the coagulation window CW after the first detection of the atrial tachycardia at time T1.

By means of the described approach, another atrial antitachycardia therapy attempt ATP4, in the example of FIG. 4, is triggered only if a change in the atrial interval exceeds a predefined margin M. Hence, it is ensured that a sufficient distance between the atrial interval at the time T5 of the prior atrial antitachycardia therapy attempt ATP3 and the atrial interval at a subsequent time T6 is present for triggering another, subsequent atrial antitachycardia therapy attempt ATP4. In this way it can be made sure that another atrial antitachycardia therapy attempt ATP4 is triggered only if chances of success in terminating the atrial tachycardia have substantially changed, hence avoiding repeated atrial antitachycardia therapy attempts at substantially unchanged conditions.

With the described approach, a classification of atrial interval values into predefined bins is avoided, hence easing the processing and allowing in particular that another, subsequent atrial antitachycardia therapy attempt is triggered only if a substantial change in the atrial interval, larger than the predefined margin M, has taken place. When classifying atrial intervals into bins, it may occur that an atrial interval is close to a boundary of a bin, making it possible that only a small change in the atrial interval may cause a reclassification into another bin and hence a triggering of another atrial antitachycardia therapy attempt.

The idea underlying the invention is not limited to the embodiments described above, but may be implemented in a different manner.

The implantable medical stimulation device may be in IPG, and ICD, or a CRT device, for example a CRT-D or CRT-P device.

The implantable medical stimulation device may, in addition to an ATP function, for example have a defibrillation function, such that after unsuccessful atrial antitachycardia therapy attempts a defibrillation shock therapy may be triggered if necessary.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical stimulation device
10 Generator device
100 Processing circuitry
11, 12, 13 Electrode lead
14, 15, 16 Pole arrangement
100 Classify atrial rhythm
110 Detect AT/AF
120 Determine atrial interval and stability information
130 ATP therapy
140 Assess ATP therapy
150 Pause (waiting period)
160 Compare atrial interval to previous atrial interval
170 Check whether atrial interval deviates from previous atrial interval by predefined margin
AA Atrial interval
ATP1 . . . ATP4 ATP therapy attempt
CW Coagulation time window
D Delay period
H Heart
M Margin
T1-T5 Time point
TH AT/AF detection threshold
VV Ventricular interval
W Waiting period

1. An implantable medical stimulation device for providing an atrial antitachycardia pacing therapy, comprising:
a generator device (comprising processing circuitry for processing cardiac sense signals to detect an atrial tachycardia and to generate stimulation signals for providing an atrial antitachycardia pacing therapy,
wherein the processing circuitry is configured to monitor a measure indicative of an atrial interval, to detect an atrial tachycardia based on said measure indicative of the atrial interval, and to trigger a first atrial antitachycardia therapy attempt if an atrial antitachycardia is detected,
wherein the processing circuitry is configured, subsequent to said first atrial antitachycardia therapy attempt, to further monitor said measure indicative of the atrial interval and to evaluate whether said atrial antitachycardia persists,
wherein the processing circuitry is configured to store a reference value based on said measure indicative of the atrial interval following said detection of the atrial antitachycardia and prior to triggering said first atrial antitachycardia therapy attempt, to compare a value of said measure indicative of the atrial interval subsequent to said first atrial antitachycardia therapy attempt to said reference value, and to trigger a second atrial antitachycardia therapy attempt if a difference between said value and said reference value is equal to or larger than a predefined margin, characterized in that said predefined margin is equal to a predefined percentage of said reference value.

2. The implantable medical stimulation device according to claim 1, wherein said predefined margin is equal to a time value in between 25 ms to 100 ms.

3. The implantable medical stimulation device according to claim 1, wherein said percentage is in a range between 10% to 50%.

4. The implantable medical stimulation device according to wherein the processing circuitry is configured to derive said measure indicative of the atrial interval by averaging values of a predefined number of atrial intervals.

5. The implantable medical stimulation device according to claim 4, wherein said predefined number is larger than 3, preferably larger than 5, more preferably larger than 10.

6. The implantable medical stimulation device according to claim 1, characterized in that the processing circuitry is configured to trigger said second atrial antitachycardia therapy attempt only if an elapsed time subsequent to said first atrial antitachycardia therapy attempt exceeds a predefined waiting period.

7. The implantable medical stimulation device according to claim 6, said predefined waiting period lies in a range between 1 minute to 20 minutes.

8. The implantable medical stimulation device according to claim 1, wherein the processing circuitry is configured to trigger said second atrial antitachycardia therapy attempt only if an elapsed time subsequent to said first atrial antitachycardia therapy attempt does not exceed a predefined coagulation time window.

9. The implantable medical stimulation device according to claim 8, wherein said predefined coagulation time window lies in a range between 20 hours to 60 hours, preferably 40 to 50 hours.

10. The implantable medical stimulation device according to claim 1, wherein the processing circuitry is configured to store the value of said measure indicative of the atrial interval at the time of triggering said second atrial antitachycardia therapy attempt as a new reference value, to compare a value of said measure indicative of the atrial interval subsequent to said second atrial antitachycardia therapy attempt to said new reference value, and to trigger another atrial antitachycardia therapy attempt if a difference between the value and said new reference value is equal to or larger than the predefined margin.

11. The implantable medical stimulation device according to claim 1, wherein the processing circuitry is configured to monitor a stability measure indicative of a stability of said atrial interval over time and to trigger an atrial antitachycardia therapy attempt based on a change in said stability measure.

12. The implantable medical stimulation device according to claim 11, characterized in that the processing circuitry is configured to classify said atrial tachycardia, based on the stability measure, as stable or unstable and to trigger an atrial antitachycardia therapy attempt if the atrial tachycardia changes from stable to unstable or vice versa.

13. A method for operating an implantable medical stimulation device for providing an atrial antitachycardia pacing therapy, comprising:
processing, using a generator device comprising processing circuitry, cardiac sense signals to detect an atrial tachycardia and generating, using the generator device, stimulation signals for providing an atrial antitachycardia pacing therapy;
monitoring, using the processing circuitry, a measure indicative of an atrial interval, detecting an atrial tachycardia based on said measure indicative of the atrial interval, and triggering a first atrial antitachycardia therapy attempt if an atrial antitachycardia is detected;
further monitoring, using the processing circuitry, said measure indicative of the atrial interval subsequent to said first antitachycardia therapy attempt, and evaluating whether said atrial antitachycardia persists;
storing, using the processing circuitry, reference value based on said measure indicative of the atrial interval following said detection of the atrial antitachycardia and prior to triggering said first atrial antitachycardia therapy attempt;
comparing, using the processing circuitry, a value of said measure indicative of the atrial interval subsequent to said first atrial antitachycardia therapy attempt to said reference value; and
triggering, using the processing circuitry, a second atrial antitachycardia therapy attempt if a difference between said value and said reference value is equal to or larger than a predefined margin,
wherein that said predefined margin is equal to a predefined percentage of said reference value.
